(19) United States
(12) Patent Application Publication
(10) Pub. No.: US 2008/0177221 A1
(43) Pub. Date: Jul. 24, 2008

(54) Apparatus to prevent applicator re-use

(75) Inventors: Donald L. Millerd, San Diego, CA (US); Alan Van Houten, Carver, MN (US); Michael T. Peterson, Lakeville, MN (US); Ryan Tetzloff, Minnetonka, MN (US)

Correspondence Address: ROPES & GRAY LLP, PATENT DOCKETING 39/41, ONE INTERNATIONAL PLACE, BOSTON, MA, 02110-2624, US

(73) Assignee: Celleration, Inc., Eden Prairie, MN (US)

(21) Appl. No.: 12/004,636

(22) Filed: Dec. 21, 2007

Related U.S. Application Data
(60) Provisional application No. 60/876,678, filed on Dec. 22, 2006.

(51) Int. Cl. A61N 7/00; A61M 35/00; A61M 37/00; A61B 17/20; A61B 17/00; A44B 21/00

(52) U.S. Cl. 604/22; 24/455; 128/897; 128/898

(57) ABSTRACT

The invention provides devices to prevent re-use of an ultrasound applicator.

### RELATED APPLICATIONS

This application claims the benefit of priority to U.S. Provisional Patent Application Ser. No. 60/876,678, filed on Dec. 22, 2006. The content of the foregoing application is hereby incorporated by reference in its entirety.

### BACKGROUND

Many devices have components designed or intended to be used only once. However, convenience or expense may motivate users to re-use such components. In certain industries, such as the medical device industry, re-use of components may undermine the safe, sterile, or effective operation of the device.

Commonly-owned U.S. Pat. No. 6,569,099 discloses an ultrasonic device and method for wound treatment, the entire contents of which are incorporated herein by reference. This patent discloses, inter alia, a device that sprays liquid particles to a wound via an applicator. The liquid particles provide a medium for propagation of the ultrasonic waves. Commonly-owned U.S. patent application Ser. No. 11/473,934, the entire contents of which are incorporated herein by reference, discloses a removable applicator nozzle for an ultrasound wound therapy device. However, although generally intended to be used only once and then discarded, the removable applicator of U.S. patent application Ser. No. 11/473,934 may be re-used, which may lead to the re-use of non-sterile applicators.

As can be appreciated, an apparatus for preventing the re-use of an applicator nozzle is desirable to prevent the use of non-sterile equipment in the treatment of wounds. The present invention provides an apparatus to prevent the re-use of an applicator nozzle that may be used in non-contact ultrasound therapy for the treatment of wounds. More generally, the present invention provides a locking device to prevent re-use of removable components of medical devices.

### SUMMARY

The present invention provides a locking device. The locking device has many possible uses. For example, the locking device can be used to prevent re-use of other devices. In certain embodiments, the locking device can be used to prevent re-use of a component of a medical device, such as a detachable nozzle, treatment head, and the like. When used in this manner, the term “locking device” is synonymous with the term “apparatus for preventing re-use”. Such devices are effective for decreasing the re-use of removable components of medical devices intended for single use.

In certain embodiments, the locking device is used to prevent re-use of an applicator nozzle, for example an applicator nozzle designed for use with an ultrasound therapy device. When used in this manner, the term “locking device” is synonymous with the term “apparatus for preventing applicator nozzle re-use”.

According to one aspect of any of the foregoing, the locking device includes a housing having a first sidewall and a second sidewall, a first arm attached to the first sidewall and having a paddle portion, and a second arm attached to the second sidewall and having a hook portion. The hook portion may be configured to hold the paddle portion in a first position, such that when the hook portion is released, the paddle portion moves to a second position. In certain embodiments, movement of the paddle portion to a second position prevents re-use of, for example, an applicator nozzle or other removable device component.

According to another aspect, the invention provides an applicator nozzle interconnected to a locking device. When used in this manner, the locking device is synonymous with an apparatus for preventing re-use.

According to another aspect, the invention provides a method for preventing re-use of an applicator.

According to another aspect, the invention provides a kit for use in methods of wound care. The kit comprises an applicator nozzle and an apparatus for preventing re-use of the applicator nozzle. The kit may optionally include instructions for use of the nozzle, sterile wipes for cleansing the nozzle prior to single use, and a warning indicating that the nozzle is designed for a single use. As packaged, the nozzle and apparatus for preventing re-use may or may not be interconnected. In certain embodiments, the contents of the kit are sterilized prior to packaging.

In certain embodiments, the kit further includes a bottle or other fluid source for use in ultrasound wound therapy. The bottle optionally includes a fluid such as, for example a saline solution. In certain embodiments, the fluid consists essentially of a saline solution, for example, the fluid does not include a medicament. In certain embodiments, the kit includes flexible tubing sized and shaped to interconnect to the applicator nozzle. Exemplary kits may include any combination of the foregoing components.

The invention contemplates operative combinations of any of the foregoing or following aspects, embodiments, or features of the invention.

### BRIEF DESCRIPTION OF THE FIGURES

The above and other features and advantages of the invention will be more fully understood by the following illustrative description with reference to the appended drawings, which may not be to scale.

FIG. 1 shows an ultrasound therapy device, an applicator nozzle, and a locking device. As depicted, the locking device is an apparatus for preventing applicator nozzle re-use.

FIGS. 2A-2G show a locking device in various positions.

FIG. 3A shows a locking device coupled to an applicator nozzle.

FIG. 3B shows an interior view of the applicator nozzle.

FIG. 4 shows a locking device coupled to an applicator nozzle. The applicator nozzle is engaged with the transducer tip of an ultrasound therapy device.

FIG. 5A shows a locking device as the applicator nozzle is being removed from an ultrasound therapy device.

FIG. 5B is a magnified view of the locking device of FIG. 5A.

FIGS. 5C-5D show an embodiment of an interference-fit mechanism.

FIG. 5E shows an interior view of the applicator nozzle and the locking device of FIG. 5A.

FIG. 5F depicts an exemplary assembly of a locking device.

FIG. 5G shows a locking device with a spring.

FIG. 5H shows the an applicator nozzle having an interference pocket.

FIG. 6A is a CAD drawing of a view of an applicator nozzle.

FIG. 7 shows a perspective view of a removable applicator nozzle that includes a nozzle, a cup, and a valve, the removable applicator nozzle being illustrated operatively attached to a transducer of an ultrasound wound therapy device and with a bottle inserted therein.

FIG. 8 shows a perspective view of the removable applicator nozzle of FIG. 7.

FIG. 9 shows a perspective view of the cup and the valve of FIG. 7.

FIG. 10 shows a perspective view of a removable applicator nozzle of an alternate embodiment partially engaged with a transducer tip of an ultrasound wound therapy device.

### DETAILED DESCRIPTION OF EXEMPLARY EMBODIMENTS

The present disclosure provides a locking device, as well as kits comprising a locking device, and methods of using a locking device to prevent re-use of removable components of a device or system. One particular use of the locking device is to prevent re-use of components of medical devices. As such, the locking device is also referred to as an apparatus for preventing re-use. When coupled to a removable component of a device, the locking device prevents the component from being repeatedly used. This is of particular importance when the risk of contamination is high, and good practice warrants that a removable component is used once but not re-used.

One particular use of the locking device of the present invention is in the field of ultrasound wound care. Certain ultrasound wound therapy devices have, as a component, a removable nozzle (often referred to as an applicator or an applicator nozzle). The ultrasound therapy device is used to deliver ultrasound energy (in the presence or absence of a liquid spray) to wounds and other patient tissue. The nozzle is the portion of the device that is positioned most closely to the patient, even when used with non-contact devices where the nozzle and other device components do not contact patient tissue (e.g., wherein ultrasound energy is delivered from a non-contact distance from patient wound tissue). Because of the proximity to wound tissue, and the risk that the nozzle will become contaminated with material from the wound, operator, or other source, it is preferable for each nozzle to be used only once. The locking device of the present invention can be used to prevent re-use of an applicator nozzle, such as applicator nozzles used with ultrasound wound therapy devices. When used in this manner, the locking device is synonymous with an apparatus for preventing applicator nozzle re-use.

The present invention provides a locking device. In certain embodiments, the locking device is an apparatus designed to prevent re-use of a removable component of another device or system. In other words, in certain embodiments, the locking device is an apparatus for preventing re-use. In certain embodiments, the locking device is an apparatus designed to prevent re-use of an applicator nozzle, for example, an applicator nozzle used with an ultrasound wound therapy device. In other words, in certain embodiments, the locking device is an apparatus for preventing re-use of an applicator nozzle.

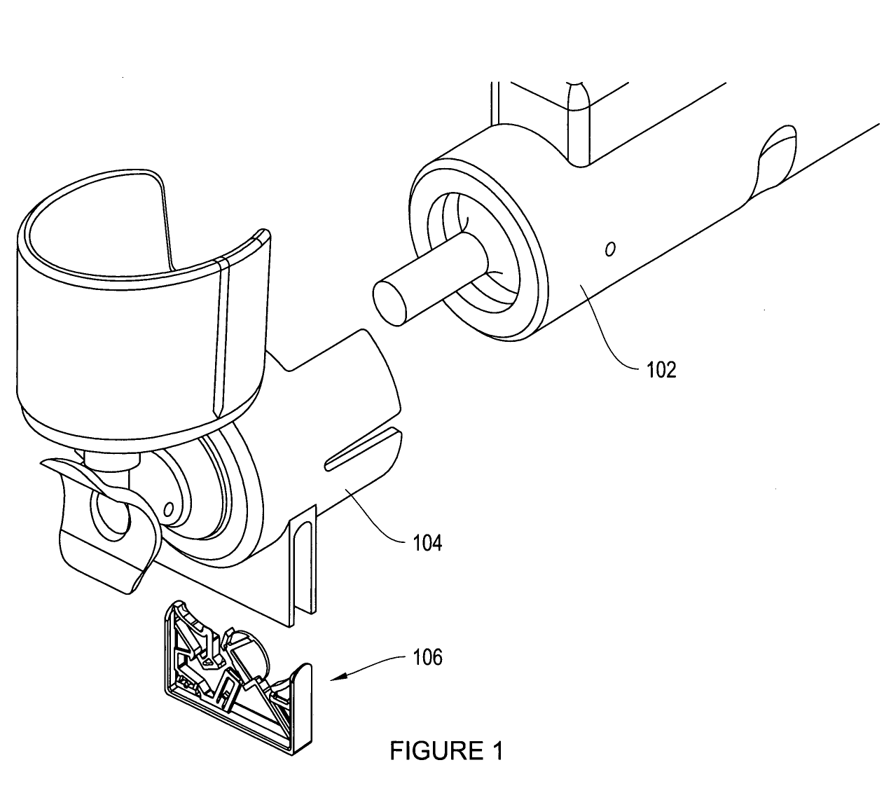

FIG. 1 shows a portion of an ultrasound therapy device 102, an applicator nozzle 104, and a locking device 106, according to an illustrative embodiment of the invention. As depicted, the applicator nozzle 104 includes a nozzle, a valve, and a cup portion and couples to the ultrasound therapy device 102 as described in U.S. patent application Ser. No. 11/473,934. This particular applicator configuration is merely illustrative, and the locking device can be used to prevent re-use of applicators having other configurations. For example, the applicator nozzle 104 may have a substantially similar configuration, excluding the cup portion. By way of further example, the applicator nozzle may have an elongated distal portion, as depicted in U.S. Patent Application Ser. No. 60/878,621, the disclosure of which is hereby incorporated by reference in its entirety.

The locking device 106 couples to the applicator nozzle 104, and is configured to allow the applicator nozzle 104 to couple with the ultrasound therapy device only one time. In this embodiment, the locking device is an apparatus that prevents re-use of the applicator nozzle. Here, the locking device can be coupled to the applicator such that, once an applicator is disengaged (following engagement) from an ultrasound therapy device, elements of the locking device change position to prevent re-engagement of the used applicator to the ultrasound wound therapy device. In other words, the locking device is designed so that an applicator can be functionally interconnected to a wound therapy device and used only one time. Once the applicator is uncoupled from the wound therapy device, the locking device prevents re-use of the applicator by preventing functional re-interconnection of the applicator to the wound therapy device.

As shown in FIG. 1, the ultrasound therapy device 102 and locking device 106 are not coupled to the applicator nozzle 104. In use, the locking device 106 is coupled to the applicator nozzle 104 before attaching the applicator nozzle to the ultrasound therapy device 102. According to one embodiment, the locking device 106 is initially configured in a ready-to-be used position (not shown) to allow the ultrasound therapy device 102 to be coupled to the applicator nozzle 104. In one embodiment, the ready-to-be-used position is a position that the locking device assumes before the ultrasound therapy device is coupled to the applicator. When the ultrasound therapy device 102 couples to the applicator nozzle 104, the locking device 106 shifts into an open position (not shown). Once the ultrasound therapy device 102 has been removed from the applicator nozzle 104, the locking device 106 shifts into a closed position and prevents re-coupling of the applicator nozzle 104 to the ultrasound therapy device 102, as discussed in greater detail below.

This particular applicator nozzle design is merely exemplary of the nozzle designs with which locking device 106 can be used. Any nozzle design suitable for use with the ultrasound therapy device can be readily configured for interconnection with the locking device 106. In use, the locking device 106 prevents re-use of an applicator nozzle, such as applicator nozzle 104. Similarly, this particular applicator nozzle is merely exemplary of a removable device component with which the locking device can be interfitted to prevent re-use.

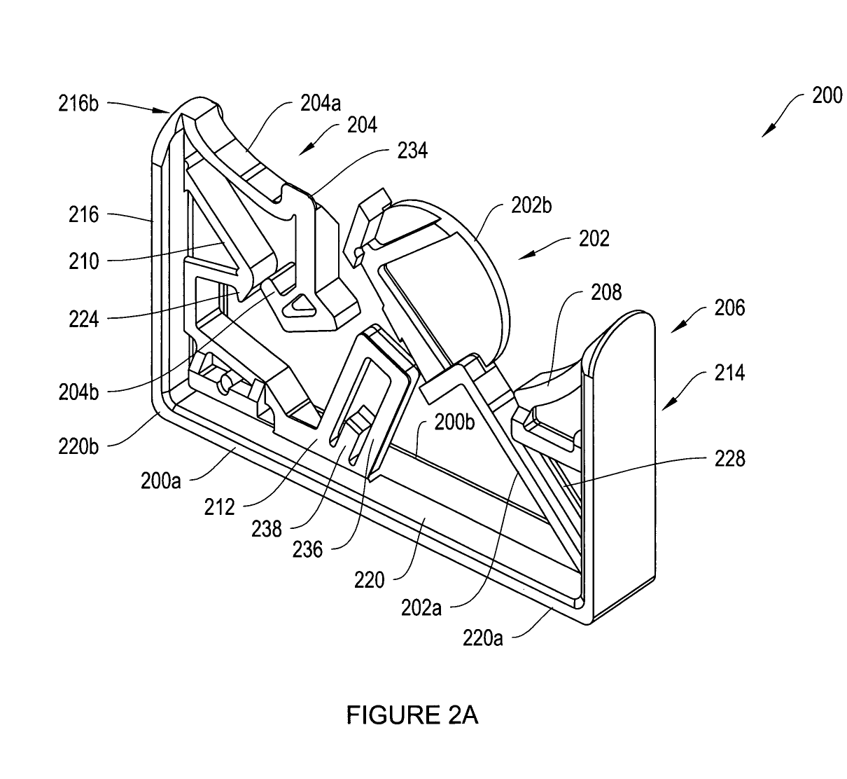

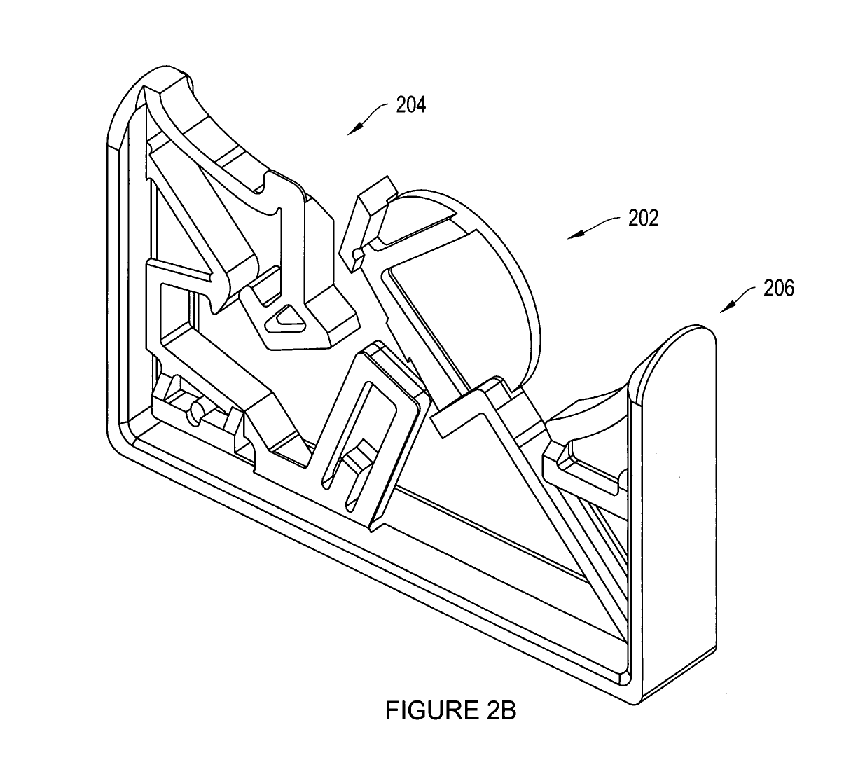

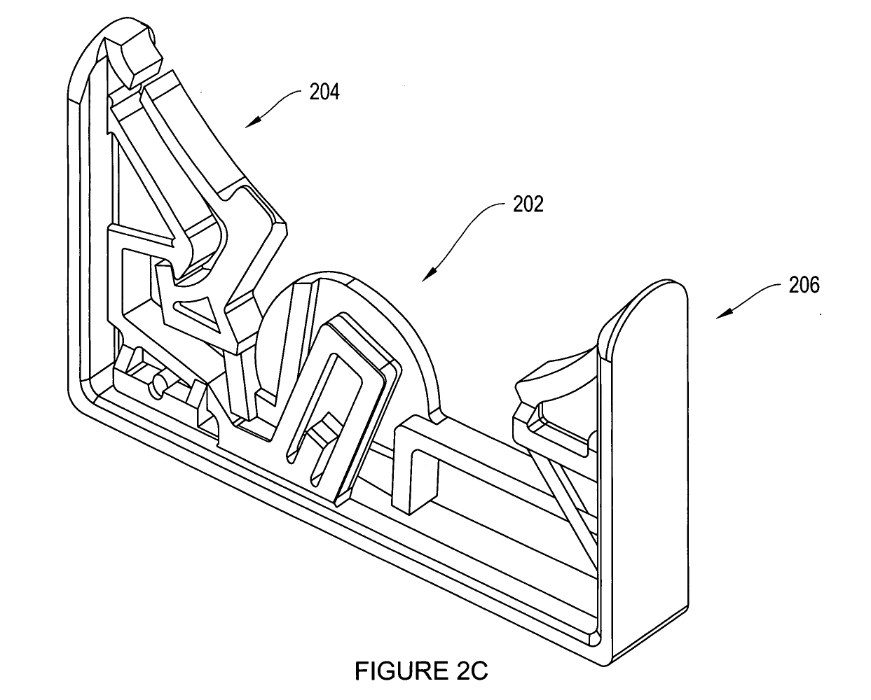

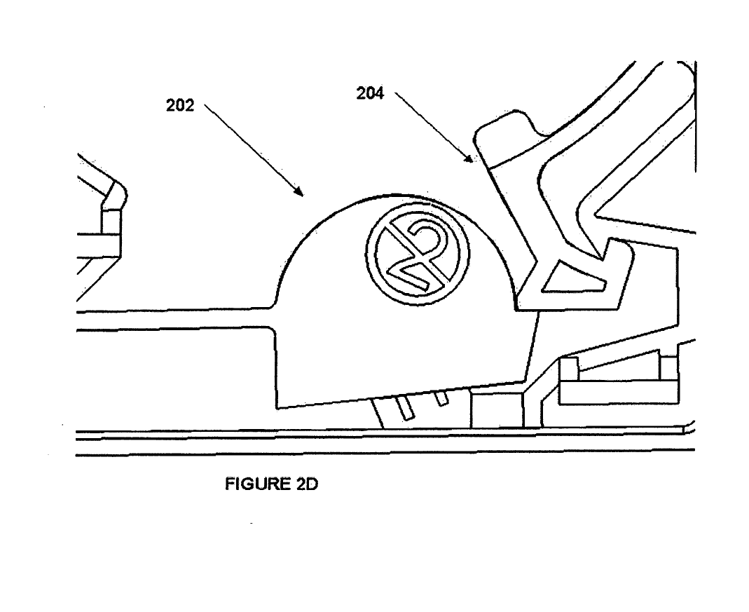

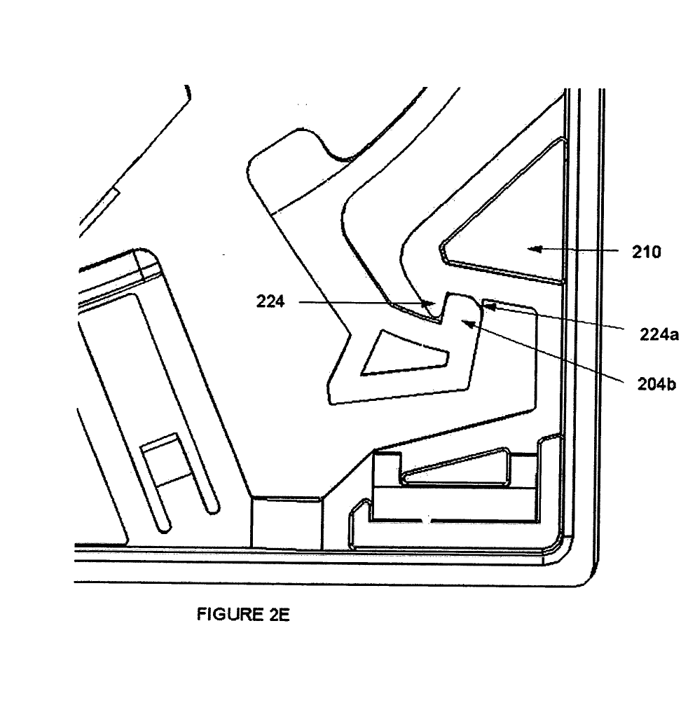

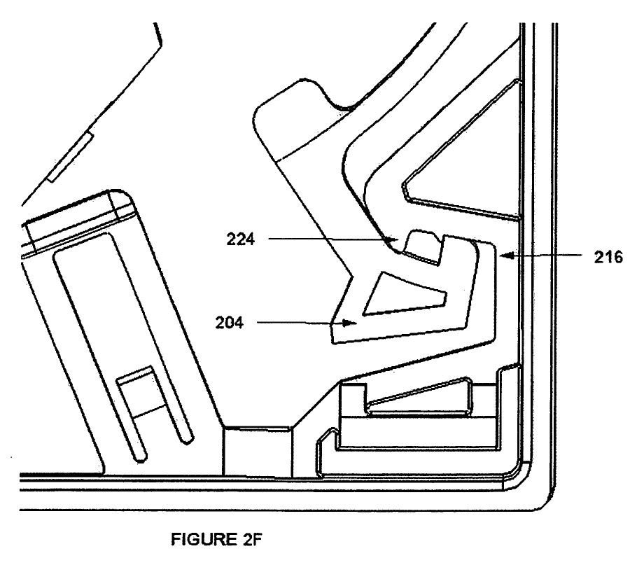

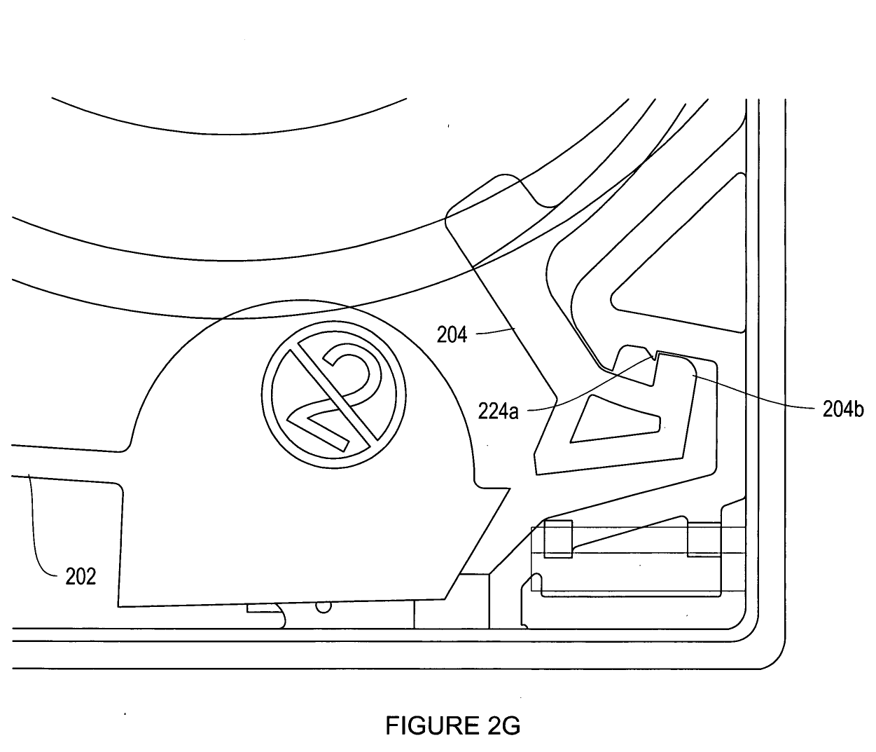

FIG. 2A shows a locking device 200 including an obstruction arm 202, a positioning arm 204, a housing 206, a first side support 208, a second side support 210, and a base support 212. The housing 206 includes first 214 and second 216 walls and a base 220. The first wall 214 extends perpendicularly from a first end 220a of the base 220, and the second wall 216 extends perpendicularly from a second end 220b of the base 220, such that the housing 206 forms a U-shape. The obstruction arm 202, the positioning arm 204, the first side support 208, the second side support 210, and the base support 212 are all attached to the housing 206 and extend inward towards the center of the U-shape. The locking device 200 has a proximal side 200a and a distal side 200b.

The obstruction arm 202 includes an obstruction arm lever portion 202a and a paddle portion 202b. As shown in FIG. 2A, the obstruction arm lever portion 202a extends toward the center of the U-shape at about a 45 degree angle from the corner of the housing formed at the intersection of the first wall 214 and the base 220. The paddle portion 202b is attached to the end of the obstruction arm lever portion 202a. As depicted, the paddle portion has a flat hemi-elliptical shape. However, other shapes are possible and similarly contemplated. The paddle portion 202b rests in a position that is about equidistant from the tops of the first 214 and second 216 walls. The obstruction arm 202 is pivotable about the point where it attaches to the first wall 214. In certain embodiments, the obstruction arm 202 may be a cantilever spring, anchored at the point where it attaches to the first wall 214.

The positioning arm 204 includes a positioning arm lever portion 204a and a hook 204b The positioning arm lever portion 204a extends inward toward the center of the housing 206 from the top end 216b of the second wall 216. The hook 204b is attached to the end of the positioning arm lever portion 204a, and extends downwards toward the base 220, back towards the second wall 216, and slightly upwards. A tab 234 extends upwards from the top surface of the positioning arm lever portion 204a. The tab 234 is positioned at the end of the positioning arm lever portion 204a just above the hook 204b, and has a width that is narrower than the width of the positioning arm lever portion 204a. The width of the tab 234 gradually narrows from the base toward the top edge, such that the tab 234 is wider at the base than at the top edge. The proximal side of the tab 234 is sloped to create the changing width, forming a cam face, while the distal side of the tab 234 has a flat surface. The positioning arm 204 is pivotable about the point where it attaches to the second wall 216. In certain embodiments, the positioning arm 204 may be a cantilever spring, anchored at the point where it attaches to the second wall 216.

The first side support 208 extends inward from the first wall 214. The first side support 208 is triangularly shaped, and includes a bottom edge 228 extending at about a 45 degree angle from the first wall 214, which aligns with the obstruction arm lever portion 202b. The first side support 208 is in the same plane as the obstruction arm lever portion 202b, and thus the bottom edge 228 of the first side support 208 prevents the obstruction arm lever portion 202b from moving upwards past the position shown in FIG. 2A.

The second side support 210 extends inward from the second wall 216. The second side support 210 is triangularly shaped, with a first side including portion of the second wall 216, a second side extending downward at an angle from the base of the positioning arm lever portion 204a, and a third side extending inward from about the middle of the second wall 216. As depicted, the second side support 210 includes a catch 224, which extends downwards toward the base 220. In some configurations, the catch 224 interfits with the hook 204b of the positioning arm 204.

The base support 212 extends inward from the base 220 toward the center of the U-shaped housing 206. The base support 212 includes a surrounding edge 236 and an inner arm 238. The base support 212 is positioned distal to and below the obstruction arm 202.

As shown in FIG. 2A, the obstruction arm 202 is in a closed position, such that, if coupled to an applicator nozzle or other removable device component, the locking device 200 would prevent, for example, the applicator nozzle from coupling with an ultrasound therapy device. To reposition the locking device 200 into an open position, the positioning arm 204 is moved downward, and hooks under the second side support 210, as shown in FIG. 2C. The hook portion 204b of the positioning arm 204 interfits with catch 224 of the second side support 210, to prevent the positioning arm 204 from releasing. Once the positioning arm 204 is in place, the obstruction arm 202 is moved downward toward the base 220, and a portion of the obstruction arm 202 is fixed in place beneath the positioning arm 204 as shown in FIGS. 2C and 2D. According to one embodiment, as shown in greater detail in the side view of FIG. 2D, the positioning arm 204 prevents the obstruction arm 202 from releasing back upwards. FIGS. 2C and 2D show the locking device 200 in an open position. As shown in FIG. 2C, the obstruction arm 202 is positioned adjacent to the base support 212.

In some embodiments, the second side support 210 has a second catch 224a to hold the hook 204b of the arm 204. As shown in FIG. 2E, the second catch 224a may be positioned from the catch 224 at a sufficient distance to fit the hook 204b between the catch 224 and the second catch 224a. In some embodiments, the second catch 224a is triangularly shaped to accommodate the rounded edge of the hook 204b. When a device, such as the ultrasound therapy device 102, is engaged with the applicator nozzle 104, the hook 204b of the arm 204 is pushed over to the side closer to the second wall 216 as shown in FIG. 2F. The arm 204 in such configuration stays locked and cannot be reset by a user. When a device, such as the ultrasound therapy device 102, is removed from the applicator nozzle 104, the obstruction arm 202 (not shown) is released. However, the repositioning arm 204 remains in place as shown in FIG. 2F. FIG. 2G shows the obstruction arm 202 being forced down, simulating a situation where a user may be attempting to circumvent the locking device (e.g., the user is attempting to re-use the removable component by circumventing the re-use prevention mechanism). However, the obstruction arm 202 cannot be held by the repositioning arm 204 because the repositioning arm 204 is fixed in a place that is out of reach of the obstruction arm 202. Thus, in this embodiment, once the applicator has been used and disengaged from the ultrasound therapy device, the repositioning arm 204 is no longer able to hold the obstruction arm 202 down. Although not required for operability of the locking device, this feature prevents the repositioning of the obstruction arm 202 from the closed position back to the open position.

According to one embodiment, the exemplary measurements of the locking device are in inches, and may vary by ±0.100 inches. The exemplary angles may vary by ±0.500 degrees. In certain embodiments, the exemplary measurements may vary by about ±0.050 inches, ±0.005 inches, or ±0.002 inches, and the exemplary angles may vary by about ±0.500 degrees, ±0.200 degrees, or ±0.100 degrees.

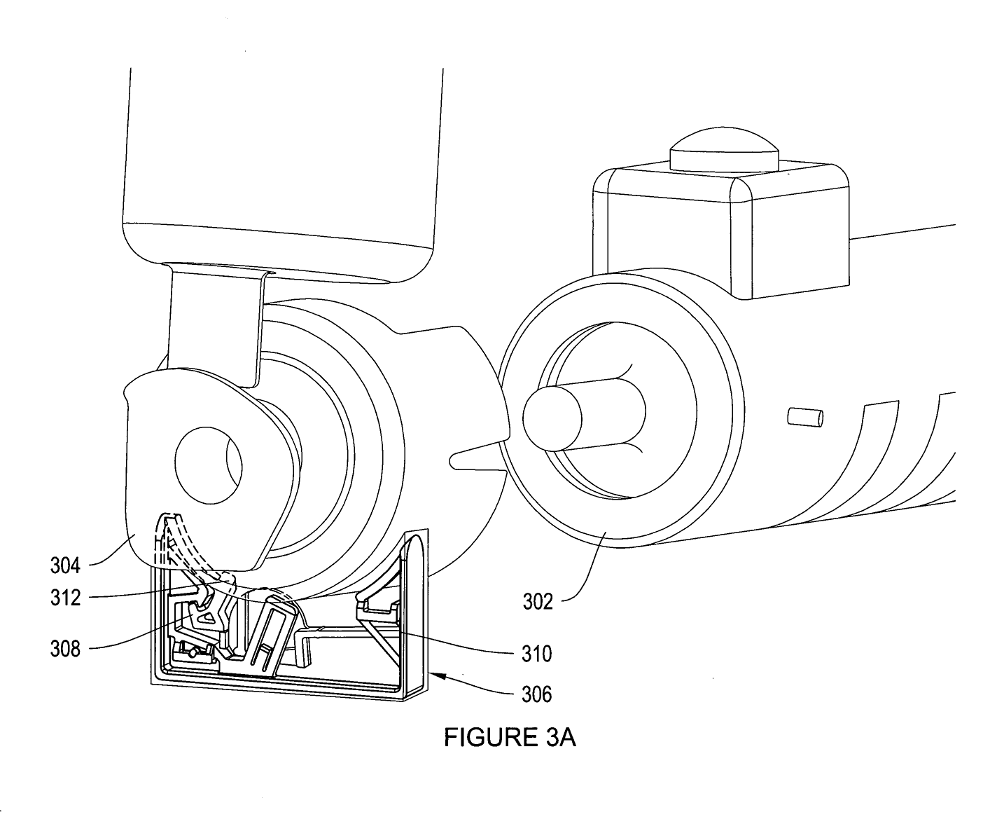

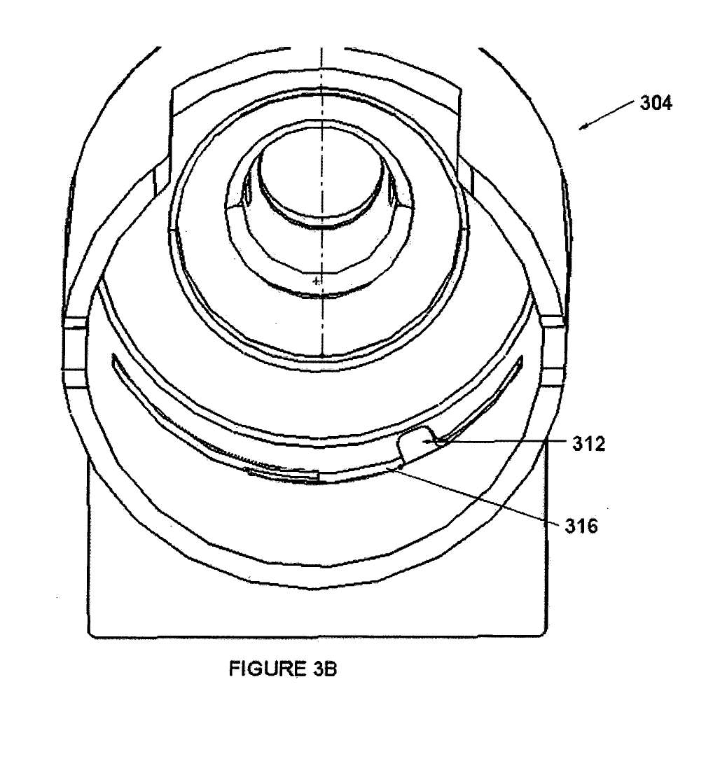

FIG. 3A depicts a locking device interfitted with an applicator nozzle. FIG. 3A shows a portion of an ultrasound therapy device 302, an applicator nozzle 304, and a locking device 306 coupled to the applicator nozzle 304. The locking device 306 is in an open position, similar to that shown in FIGS. 2C and 2D. Note that the elements of the locking device 306 which create the upper edge of the device, including the positioning arm 308 and the first side support 310 have a convex curved contour such that they interfit with the rounded edge of the applicator nozzle 304. The tab 312 of the positioning arm 308 extends upward through an opening 316 in the applicator nozzle 304, as shown in the interior view of FIG. 3B. In this configuration, the applicator 304 is new and ready to be used. The tab 312 of the positioning arm 308 is holding the obstruction arm down (not shown).

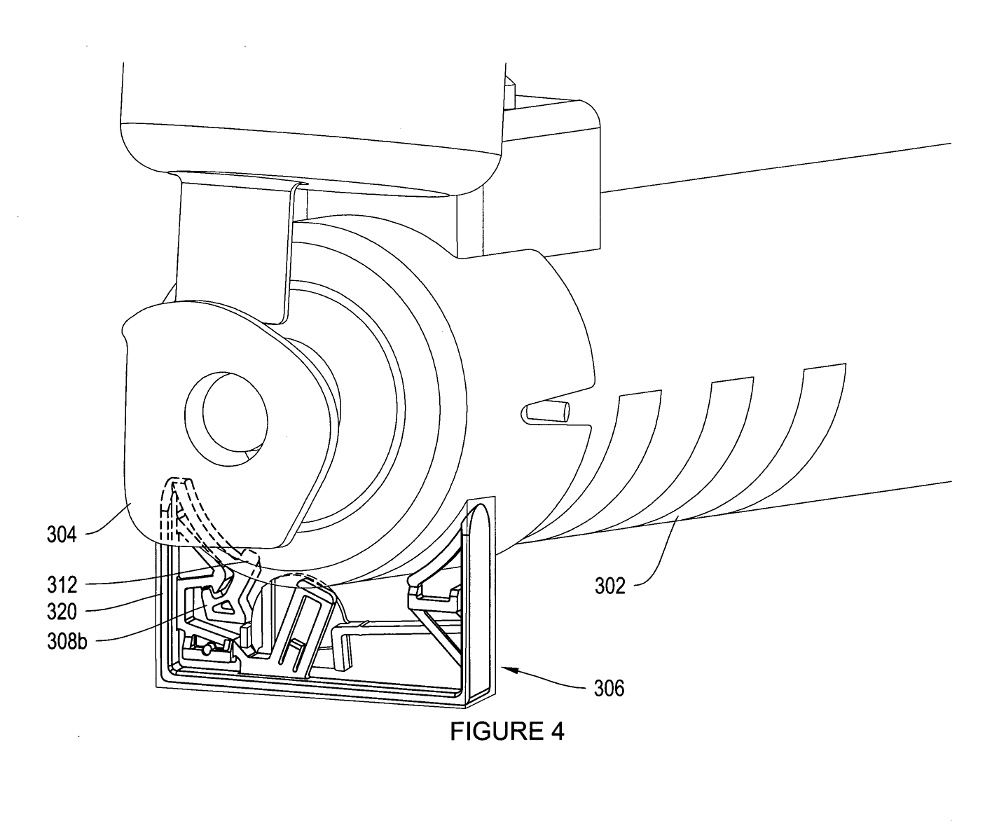

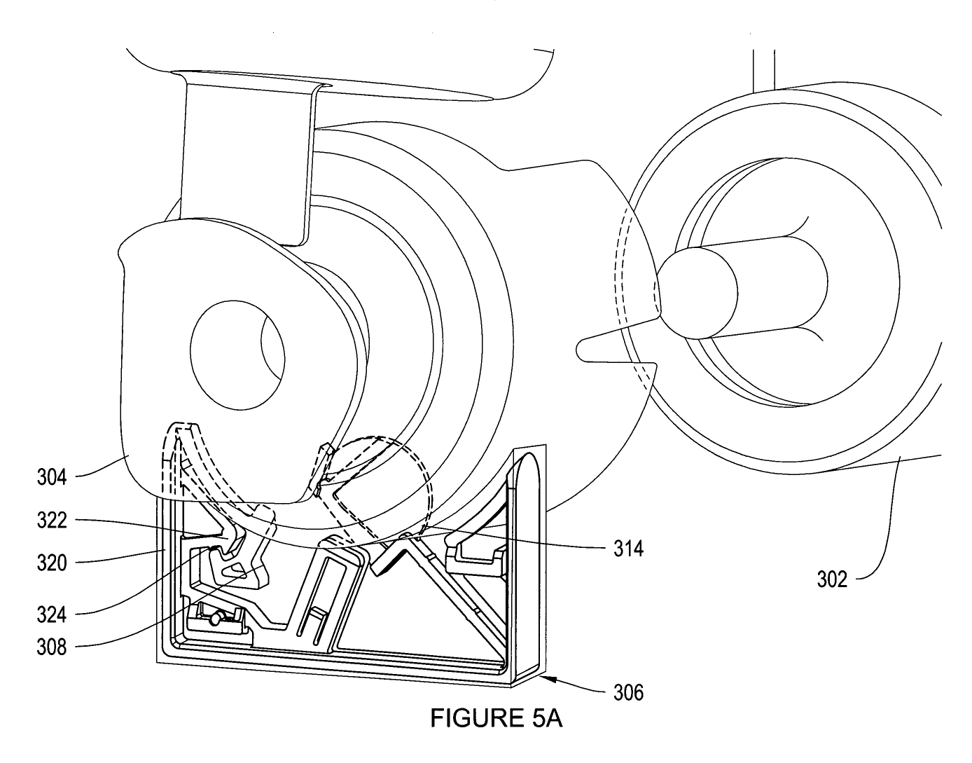

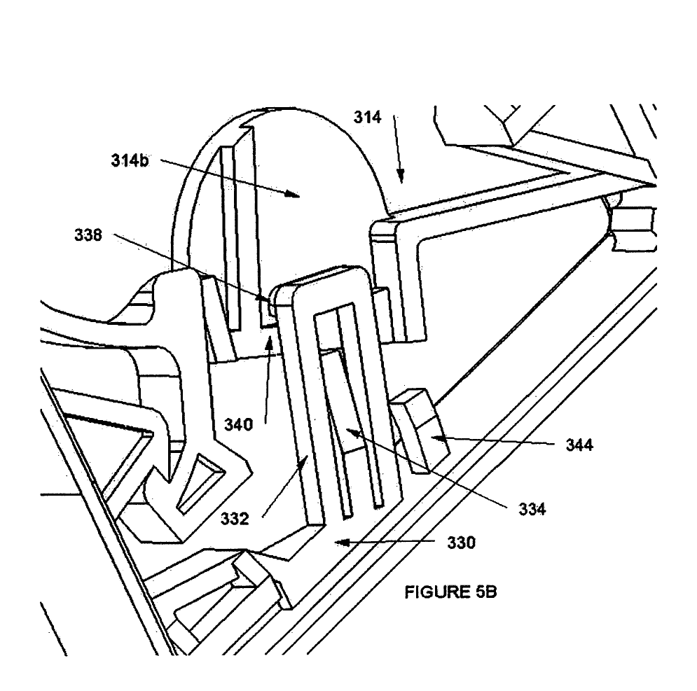

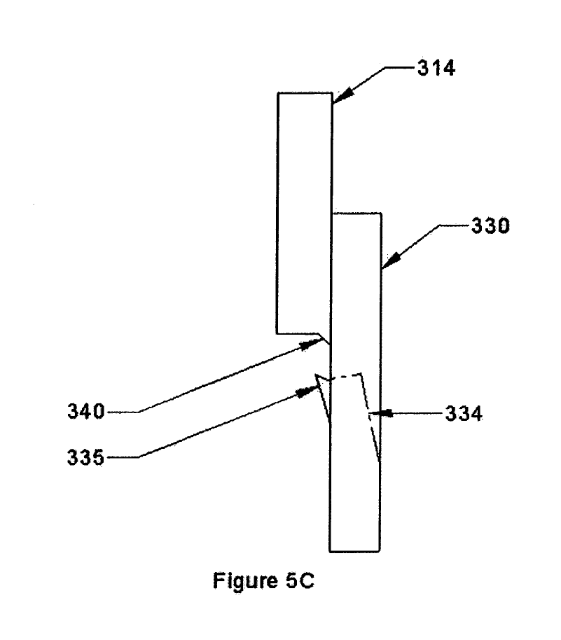

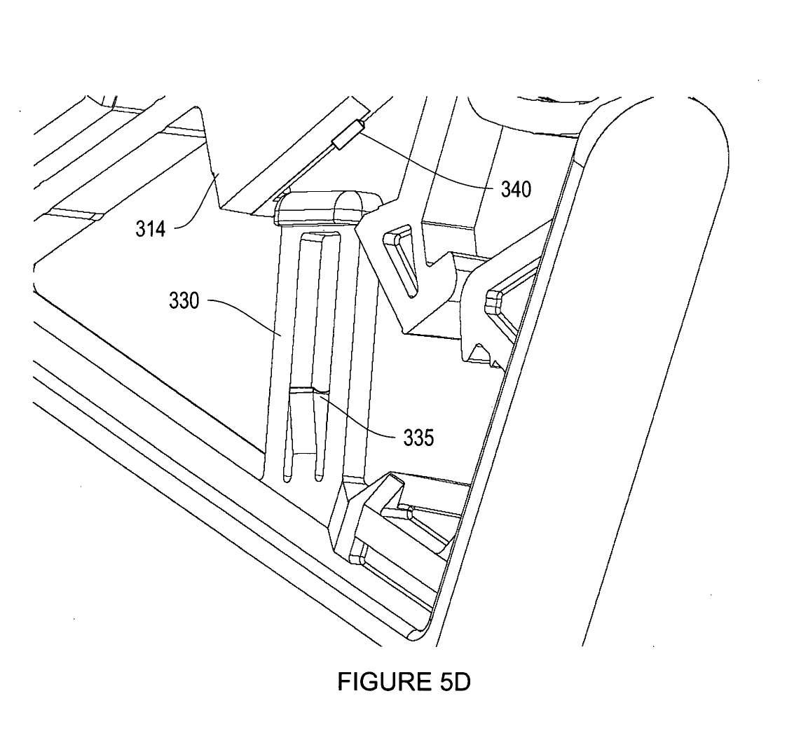

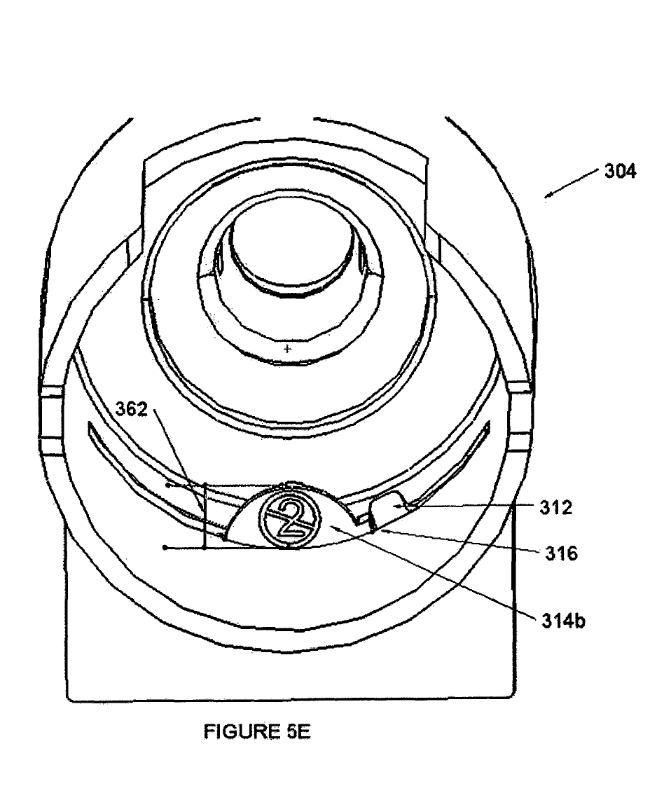

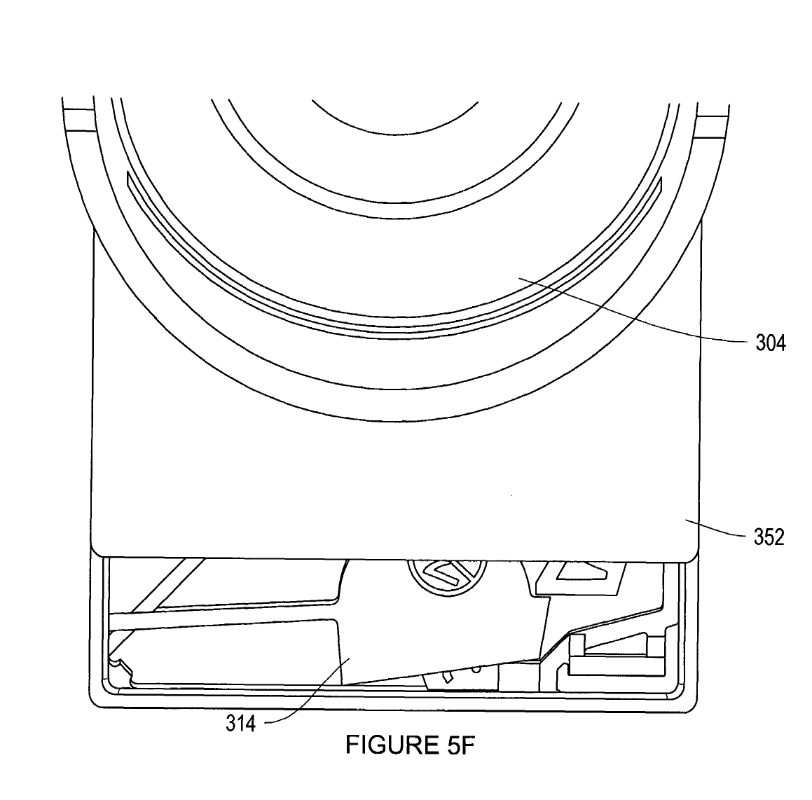

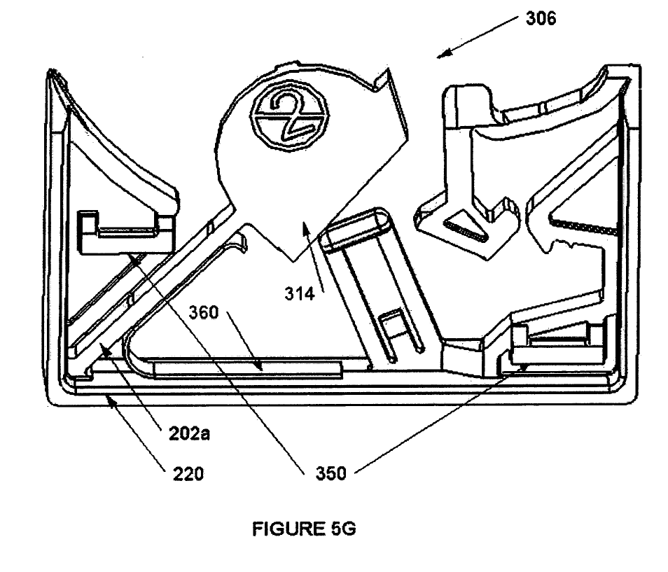

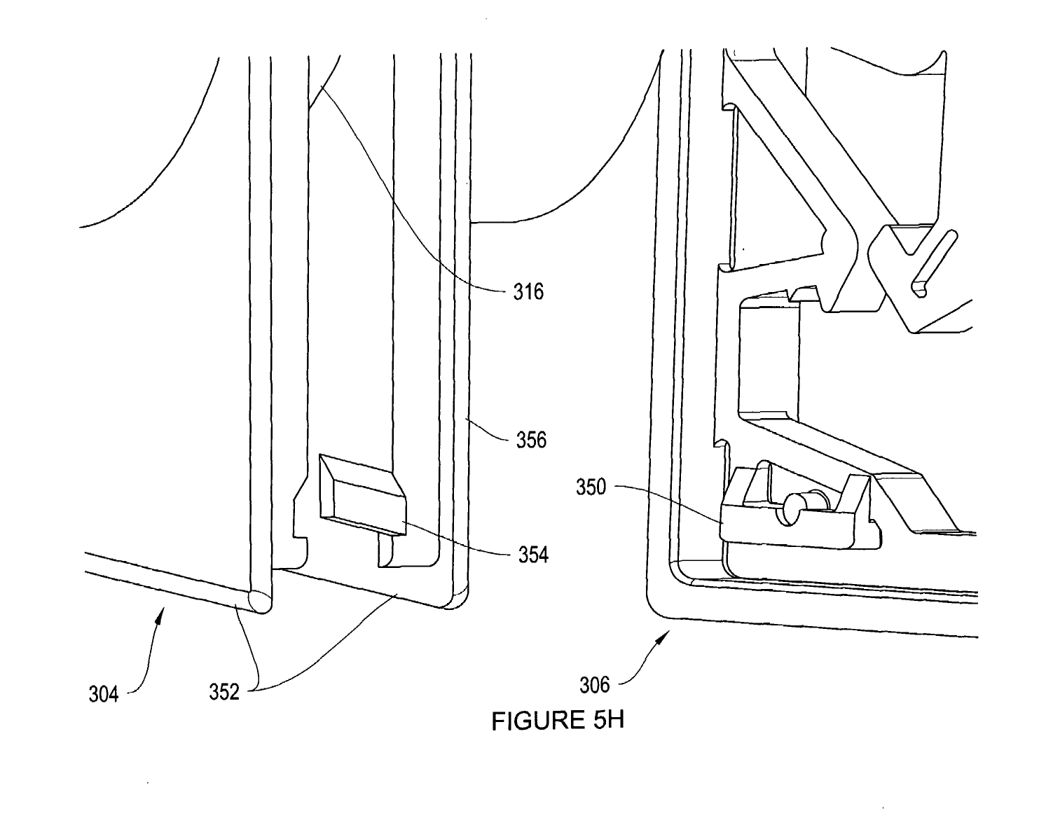

FIG. 4 shows the ultrasound therapy device 302 coupled to the applicator nozzle 304. As shown in FIG. 4, when the ultrasound therapy device 302 is inserted into the applicator nozzle 304, the ultrasound therapy device 302 pushes the tab 312 of the positioning arm 308 downwards, thereby moving the hook portion 308b of the positioning arm 308 towards the second wall 320 of the locking device 306 and releasing the obstruction arm from its open position. The released obstruction arm is held by the therapy device 302 when the device is in use. Following the disengagement of the ultrasound therapy device 302 from the applicator nozzle 304, the obstruction arm is released through the opening 316 as shown in FIGS. 5A and 5E.

FIG. 5A shows the ultrasound therapy device 302 being removed from (e.g., disengaged from) the applicator nozzle 304. As shown in FIG. 5A, when the outer edge of the ultrasound therapy device 302 moves proximally past the locking device 306, the positioning arm 308 and the obstruction arm 314 are released. Because the positioning arm 308 had been pressed against the second wall 320 of the locking device, when it is released, it springs back inward past the catch 324 of the second side support 322, and then up to a closed position, as shown in greater detail in FIG. 5B. The obstruction arm 314 is simultaneously released, and springs up past the positioning arm 308 and through the opening in the applicator nozzle 304 to the closed position. In this position, the obstruction arm 314 prevents the applicator nozzle 304 from being re-coupled to an ultrasound therapy device 302, and thus prevents re-use of the applicator nozzle 304.

FIG. 5B is a magnified view of the locking device 306 in the closed position following coupling of an ultrasound therapy device to the applicator 304. As shown in FIG. 5B, the upper portion of the surrounding edge 332 of the base support 330 includes a protrusion 338. The protrusion 338 extends over a projection 340 on the lower portion of the paddle portion 314b of the obstruction arm 314, thereby limiting the upward movement of the obstruction arm 314, and preventing the obstruction arm 314 from springing any further upwards. FIG. 5B also shows the inner arm 334 of the base support 330, which, following removal of the ultrasound therapy device, shifts distally, such that it is positioned underneath a bottom edge of the paddle portion 314b of the obstruction arm. The inner arm 334 thus prevents the obstruction arm 314 from being moved back downward, thereby preventing the possibility of repositioning the locking device 306 back to the open position. In some embodiments, 0.010 inches of interference contact between the inner arm 334 of the base support 330 and the projection 340 of the obstruction arm 314 may be sufficient to prevent the obstruction arm 314 from resetting to the open position. In some embodiments, the interference contact may range from 0.012 inches to 0.015 inches or it may be greater than 0.015 inches to prevent a user from resetting the obstruction arm 314 forcefully. The locking device may also includes a second base support 344. According to one embodiment, if the inner arm 334 of the base support 330 were to break or otherwise not function to prevent repositioning of the locking device 316 to a open position, the second base support 344 further prevents the obstruction arm 314 from being pushed back downward and thus prevents repositioning of the locking device 316.

FIGS. 5C and 5D show alternative embodiments of the inner arm 334 having a tapered edge 335. The inner arm 334 has a larger width at the top compared to the bottom of the arm. The projection 340 of the obstruction arm 314 is also tapered to create an interlock connection with the tapered edge 335. In this configuration, interference contact of about 0.008 inches to about 0.010 inches between projection 340 and the tapered edge 335 of the inner arm 334 may be sufficient to prevent the user from resetting the obstruction arm 314 to the open position. Although not required for operability, when present, this feature helps prevent circumvention of the locking device (e.g., helps prevent purposeful re-use).

FIG. 5E shows an interior view of the applicator 304 with the locking device 306 in the closed position following removal of the ultrasound therapy device from the applicator 304. As shown in FIG. 5E, the paddle portion 314b of the obstruction arm extends through the aperture 316 and prevents insertion of an ultrasound therapy device (prevents engagement of the applicator with the ultrasound therapy device). FIG. 5E depicts a protrusion distance 362 that is sufficient to prevent the ultrasound therapy device from re-coupling to the applicator 304. The protrusion distance 362 may be in the range of about 0.100 inches to 0.250 inches.

FIG. 5F shows the obstruction arm 314 in a ready-to-be used position. The obstruction arm 314 is held by the positioning arm 308 (FIG. 3A). FIG. 3B also shows an embodiment of a ready-to-be-used position showing a tab 312 of the positioning arm 308 (FIG. 3A). The obstruction arm 314 remains in the position depicted in FIG. 5F through the duration of its shelf life. In some embodiments, it is possible that during the duration of its shelf life, the obstruction arm 314 fixed in the ready-to-be-used position loses elasticity over time. To prevent a possible loss in elasticity from impeding the function of the locking device, in some embodiments, a spring is included in the device. When present, the spring helps lift the obstruction arm 314 to a distance that is sufficient to prevent re-coupling of the ultrasound therapy device to the applicator, as shown in FIG. 5E. FIG. 5G shows a spring 360 that is positioned between the obstruction arm lever portion 202a (FIG. 2) and the base 220 (FIG. 2) of the locking device 306. After the ultrasound device 302 is removed from the applicator nozzle 304, the spring 360 lifts the obstruction arm 314 through the aperture 316 of the applicator nozzle 304 to prevent re-use of the applicator. The spring may also prevent the obstruction arm 314 from collapsing to a closed position. Exemplary types of spring include coil springs, torsion springs, leaf springs, and V-springs. The spring can be made of metal, such as hardened steel or stainless steel. In certain embodiments, the spring is made using corrosion resistant materials, such as non-ferrous metals. In certain embodiments, the spring is made from or contains an elastic material such as rubber or silicon.

According to one embodiment, the locking device 306 is permanently attached to the applicator nozzle 304. The locking device 306 may snap-fit with the applicator nozzle 304. In one embodiment, the locking device 306 and applicator nozzle 304 have an interference-fit between one another. The locking device 306 has tapered protrusions 350 (FIG. 5G) and the applicator nozzle 304 has tapered pocket 354 (FIG. 5H) for receiving the tapered protrusions 350. The locking device 304 is assembled to the applicator nozzle 304 by placing the locking device 306 into the space between the two parallel walls 352 (FIG. 5H) of the applicator nozzle 304. In some embodiments, the locking device 306 is glued or heat-bonded to the applicator nozzle 304. For example, a bonding solvent such as cyclohexanone may be applied along the edge 356 shown in FIG. 3D. When used with other removable components, the locking device may be similarly interfitted with the removable component.

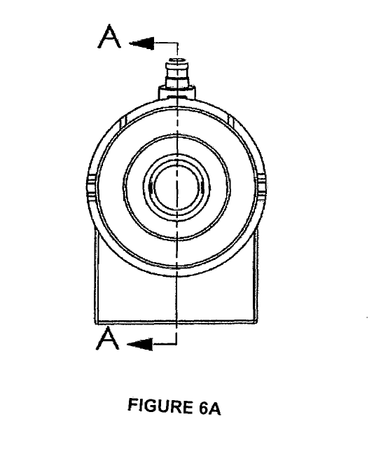

FIG. 6A is a CAD drawing showing a front view of the applicator nozzle. According to one embodiment, the exemplary measurements of the features of the applicator nozzle are in inches, and may vary by ±0.100 inches. The exemplary angles may vary by ±0.500 degrees. In certain embodiments, the exemplary measurements may vary by about ±0.050 inches, ±0.005 inches, or ±0.002 inches, and the exemplary angles may vary by about ±0.500 degrees, ±0.200 degrees, or ±0.100 degrees.

According to various embodiments, the locking device and applicator nozzle may be constructed from any selected material or combination of materials, including, for example, plastics, thermoplastics, polymers, polycarbonates, and metals. One exemplary material is GE Lexan polycarbonate HPS4. Another exemplary material is Bayer Bayblend® FR110 polycarbonate/ABS resin with Baystate Polymer L8623. The materials may include white colorant, blue colorant, or any other selected colorant. In certain embodiments, the applicator nozzle and the locking device are constructed from the same material. In other embodiments, the applicator nozzle and the locking device are constructed from different materials. In certain embodiments, the applicator nozzle and the locking device are gamma irradiated or otherwise sterilized prior to packaging. In other words, the applicator nozzle and the locking device are sterilized such that they are sterile when packaged and sold, prior to their use.

According to various embodiments, in use the locking device and applicator nozzle are interconnected. However, the locking device and applicator nozzle may be constructed and sold as an interconnected unit, or may be constructed separately and later interconnected. Such separate construction and interconnection includes, for example, retro-fitting of previously fabricated applicators.

In another aspect, the invention provides a kit containing an applicator nozzle and a locking device. In certain embodiments, the kit contains an applicator nozzle interconnected to a locking device. In other embodiments, the kit contains an applicator nozzle and a locking device that have not yet been interconnected. The components of the kit can be sterilized prior to packaging, such that the applicator nozzle and locking device are sterile prior to use.

In certain embodiments, the kit further contains one or more additional components. Exemplary additional components include, but are not limited to, instructions for use, a warning label that reminds the user that the applicator is intended for single-use, sterile wipes, flexible tubing, a bottle or other fluid container (with or without fluid).

As noted above, U.S. application Ser. No. 11/473,934 provides a detailed description of an ultrasound wound therapy device suitable for use in non-contact wound therapy, including a detailed description of exemplary ultrasound transducer and applicator nozzle designs. These designs and features of devices and methods for non-contact ultrasound wound therapy are exemplary of nozzle designs with which the apparatus described in the instant application can be used. The invention contemplates suitable combinations of any of the aspects and embodiments disclosed in the present application with the aspects and embodiments disclosed in application Ser. No. 11/473,934, filed Jun. 23, 2006, as well as aspects and embodiments describing additional nozzle designs disclosed in Application Ser. No. 60/878,621, filed Jan. 4, 2007. Application Ser. Nos. 11/473,934 and 60/878,621 are incorporated by reference in their entirety.

The above description of kits equally applies to a locking device designed for interconnection with a component of another type of device.

To further illustrate, applicants provide below a brief discussion of the methods and devices for non-contact ultrasound therapy described in the above referenced co-pending applications. Note that the particular applicator configurations described in these applications and summarized below are merely exemplary of the removable device components that can be interfitted with the locking device of the present invention.

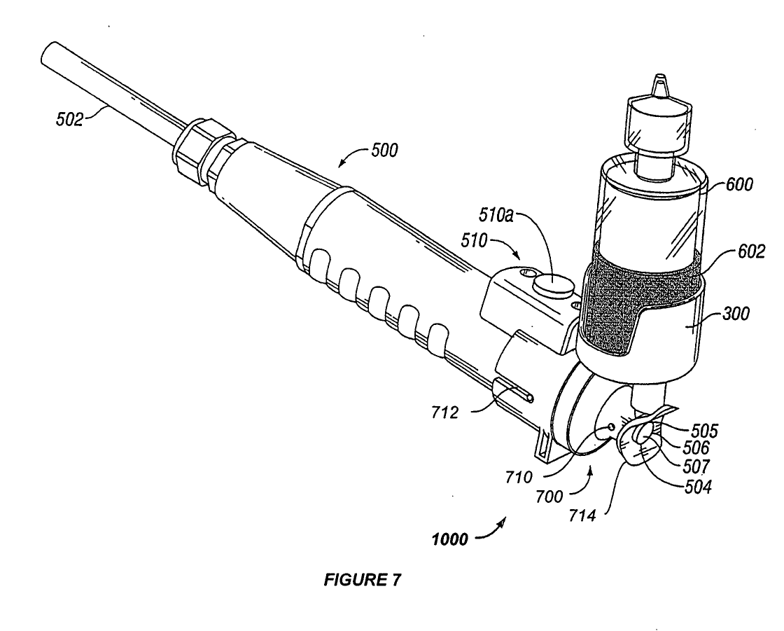

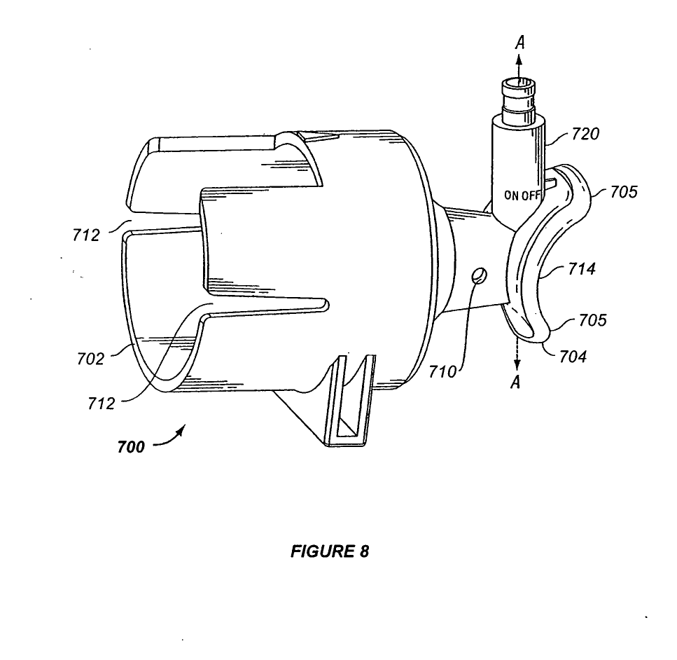

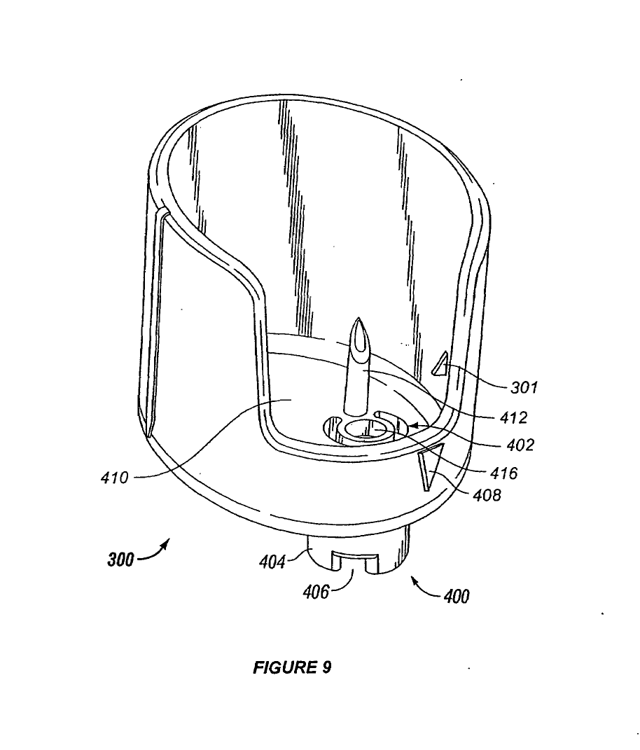

FIG. 7 depicts removable applicator 1000 including a transducer assembly 500 and an applicator nozzle 700 (FIG. 8). As depicted, applicator nozzle 700 includes a cup 300 (FIG. 9) and a valve 400 (FIG. 9), although these cup and valve features are not necessarily present in other embodiments of the applicator.

Referring to FIG. 8, as depicted, the nozzle 700 includes a proximal portion 702, a distal portion 704, a plurality of alignment slots 712, a distal opening 714, and a valve interface 720. The portion of the nozzle 700 that extends the farthest distally is distal tip 705.

It is envisioned for the applicator 1000 to be designed for use with an ultrasound wound therapy device, such as the device described in U.S. Pat. No. 6,569,099 or U.S. application Ser. No. 11/473,934, the entire contents of which are incorporated herein by reference.

An exemplary ultrasound wound therapy device includes a transducer assembly 500 operatively connected to a generator (not shown). As described herein, the ultrasound wound therapy device may further include an applicator 1000 that can be interconnected to (engaged with) the transducer assembly. Briefly, the generator includes the components necessary to supply power to the transducer assembly, and also contains a graphical user interface (GUI) for displaying information helpful to the operator. The generator consists of three major functional sections: the AC MAINS, the main board, and the GUI board. The local AC MAINS is connected to an appliance inlet with a hospital grade detachable power cord. The appliance inlet is a power entry module listed for medical applications. In certain embodiments, the appliance inlet is a power entry module with an 115V/230V voltage selection, and is designed to operate on 115 Vac and 60 Hz (e.g., for operation in North America) or 230 Vac and 50 Hz (e.g., for operation in Europe).

The MAIN board converts the secondary output voltage from the MAINS transformer to the low voltage power rails for the internal electronics and the drive voltage for the drive electronics to the transducer assembly. The MAIN board contains a microprocessor that controls, measures, and monitors the drive electronics. The transducer assembly connects to the MAIN board. The microprocessor, referred to as the engine, monitors the performance of the system and communicates the information to a second microprocessor located on the GUI board. In certain embodiments, the engine communicates to the second microprocessor via a RS-232 communication link. In certain embodiments, the electronics drive the ultrasound portion of the drive electronics with a push-pull converter that has a feedback loop with a Phase Locked Loop (PLL) to track the center frequency of the ultrasound components.

The GUI board provides the graphical user interface for the operator. A custom membrane switch panel with, for example 6 keys, allows the operator to select the functions and operating parameters of the system. A purchased graphical LCD display, connected to the GUI board, can be used to display information to the operator. For example, information about the system's status, mode of operation, and treatment time can be displayed via the GUI. The GUI may have a back light generator for the LCD on it. The GUI microprocessor runs the system by controlling the human interface and running the various algorithms to control the operation of the system. For example, a treatment algorithm can be run on the GUI microprocessor. In certain embodiments, the ultrasound wound therapy device may include one or more of a timer to record total treatment time, a timer to count-down from a selected treatment time to zero, and an alarm to indicate that the total treatment time has elapsed or that there is a problem with some component of the device.

Now referring to FIG. 9, as depicted, the applicator includes a cup 300 which includes a puncturing device 412, a lower portion 410 with an aperture 416 extending therethrough, and may include an alignment structure 308. Note, however, that other applicator configurations do not include a cup portion. Rather, in certain embodiments, fluid can be delivered to the nozzle via flexible tubing or other means, thereby obviating the need for a cup portion.

When present, the cup 300 may be designed to hold at least a portion of a bottle 600 (FIG. 7) therein. The bottle 600 generally holds a fluid 602, which may be saline. The fluid may alternatively be sterile water or some other isotonic or hypertonic solution or combination of solutions. The fluid may consist entirely or essentially of the saline or other similar solution, or the fluid may optionally include a therapeutic drug. The fluid may optionally be sterilized. The cup 300 may also include structure, such as indent(s) 301, on the lower inside surface of the bottle 600 as shown by FIG. 9 for enhancing the grip and fit of the bottle 600 within the cup 300. When included, indent(s) 301 are configured for damaging the bottle 600 upon removal of the bottle 600 from the cup 300, thereby preventing reuse of the bottle 600.

The valve 400 is also illustrated in FIG. 9. The valve 400 includes an upper portion 402, a lower portion 404 and a slot 406. The valve 400 selectively allows the fluid 602 from the bottle 600 to pass therethrough and towards the nozzle 700. In certain embodiments, it is envisioned for the valve 400 to be separate or removable from the applicator 1000. For example, a removable valve may be designed to disengage from the applicator when the applicator is detached from the remainder of the ultrasound wound therapy device following use. Such a design and valve configuration could be used to prevent reuse of an applicator. In other embodiments, the valve 400 is not separate or removable, but rather is included and integrated with the cup 300.

Referring to FIGS. 7-10, in the depicted example, the nozzle 700, the cup 300 and the valve 400 mechanically engage with one another to form the applicator 1000. Specifically, the lower portion 404 of the valve 400 fits over the valve interface 720 of nozzle 700; the upper portion 402 of the valve 400 fits into the aperture 416 of the cup 300. When mechanically engaged, the cup 300 is capable of turning approximately 90° with respect to an axis A-A, as defined by the valve interface 720 (FIG. 8). Turning the cup 300 adjusts the valve 400 from a closed position where the fluid 602 cannot flow through, to an open position which provides a passage for the flow of the fluid 602. Turning the cup 300 back towards its original position closes the valve 400.

The applicator 1000 is mechanically connectable with a transducer assembly 500 of an ultrasound wound therapy device, hereinafter referred to as a transducer assembly. When activated, the transducer assembly 500 produces ultrasonic waves having a frequency of about 1 kHz to about 10,000 MHz. Preferably, the transducer assembly produces low frequency ultrasonic waves of about 10-100 kHz, about 20-60 kHz, about 20-30 kHz, or about 40-50 kHz. The ultrasonic waves deliver ultrasonic energy to a wound surface, including below the wound surface, via a spray which acts as the coupling agent for the ultrasonic energy as further described below. The ultrasonic energy provides bactericidal, therapeutic and other effects for decreasing the healing time for the wound as disclosed by U.S. Pat. No. 6,569,099, the entire contents of which are incorporated herein by reference. Without being bound by theory, the liquid spray delivered to the wound may also have bactericidal, therapeutic, and other effects on wound healing at the surface of and/or below the surface of the wound. In use, ultrasound energy emitted from the transducer and a fluid spray produced when fluid is dripped on a face of the transducer are delivered to a wound.

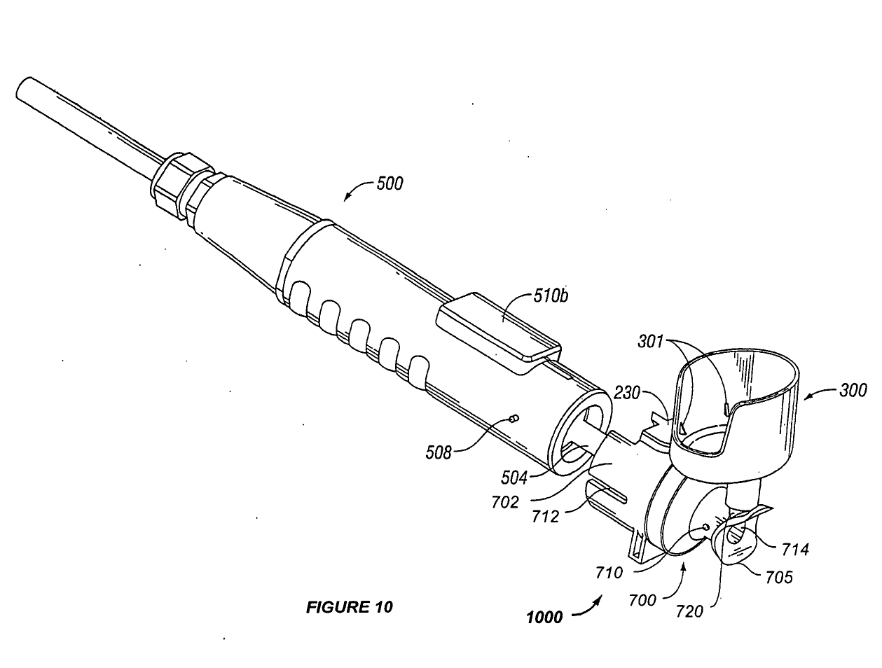

Specifically, the proximal portion 702 of the nozzle 700 slides over a distal portion 504 of the transducer assembly 500. The plurality of aligning slots 712 (illustrated as two slots) of the nozzle 700 engage with a plurality of aligning pins 508 (FIG. 10) of the transducer assembly 500. When connected, the distal end 506 of a tip 505 of the transducer assembly 500 may extend distally of the distal opening 714 of the nozzle 700 but not to a location that is distal of the tip 705 of the nozzle 700. That is, when the transducer assembly 500 is inserted through the applicator 1000, the distal end 504 of the transducer assembly 500 extends between the distal opening 714 and the distal tip 705 of the nozzle 700, such that the distal dip 705 of the nozzle 700 is coaxially disposed about the distal end 504 of the transducer assembly 500.

In use, the cup 300 is inserted onto the “valve” 400 (as shown in FIG. 9) and the valve 400 is inserted onto the valve interface 720 of the nozzle 700. The transducer assembly 500 is then aligned and coupled with the nozzle 700, via aligning slots 712 and alignment pins 508. The distal end 506 of the transducer assembly 500 is inserted through the proximal portion 702 of the nozzle 700, continues through the distal portion 704 of the nozzle 700, and out through the distal opening 714 of the nozzle 700. The bottle 600 may then be placed into the cup 300. Upon insertion of the bottle 600 into the cup 300, the puncturing device 412 of the cup 300 punctures a hole in the bottle 600. The aligning structure 308 may assist the user in properly positioning the bottle 600 in the cup 300. In certain embodiments, it is envisioned for the bottle 600 to be inserted into the cup 300 prior to the applicator 1000 being coupled with the transducer assembly 500. The insertion of the cup 300 and valve 400 into the nozzle 700, the coupling of the applicator 1000 and the transducer assembly 500, and the insertion of the bottle 600 into the cup 300 allow the applicator 1000 to be utilized vis-a-vis the transducer assembly 500.

The separation distance between the free end surface of the transducer tip 705 (FIG. 8) and the surface or object to be sprayed may be a non-contact distance of at least 0.1 inches (2.5 mm). Preferably, the separation distance is from about 2.5 mm to about 51 cm, more preferably, from about 15 mm to about 25 mm. In certain embodiments, the applicator nozzle extends distally beyond the transducer tip. Such a design has numerous benefits including the prevention of inadvertent patient or operator contact with the transducer tip. The non-contact distance can similarly be described as the distance between the distal-most edge 705 of the nozzle 700 and the surface or object to be sprayed. In certain embodiments, the non-contact distance from the distal-most edge 705 of the applicator nozzle 700 is at least about 5 mm. In other embodiments, the non-contact distance from the distal-most edge 705 of the applicator nozzle 200 is from about 5 mm to about 15 mm.

In a particularly useful embodiment, the valve opening is appropriately sized to allow a desired amount of fluid 602 to pass therethrough such that the fluid 602 that drips onto the tip 505 of the transducer assembly 500 can wrap around the circumference of the tip 505. Such an effect is known as the Babaev effect, or vacuum effect, and creates a capillary action that wicks or applies the fluid 602 around the circumference of the tip 505 of the transducer assembly 500.

The fluid 602 to be sprayed and provided within the bottle 600 can be any appropriate carrier, such as saline, water (regular or distilled), or oil to be applied to tissue, such as a vegetable, peanut, or canola oil, optionally with a soluble pharmaceutical (e.g., an antibiotic), antiseptic, conditioner, surfactant, emollient, or other active ingredient. The fluid 602 can also be a combination of two or more fluids and/or substances having microscopic particles, such as powder and the like. Exemplary fluids include, but are not limited to, sterile water, saline solution, oil, oxygenated water, or other isotonic or hypertonic solutions. Exemplary fluids may, in certain embodiments, further include drugs (e.g., therapeutic agents) such as antibiotics, anti-fungals, anti-virals, growth factors, analgesics, narcotics, and the like, formulated in any of the foregoing fluids or in other pharmaceutically acceptable fluids appropriate for the formulation of the particular drug. However, in certain embodiments, the fluid does not include a drug. The fluid may be sterilized so that, in use, a spray of a sterile solution can be administered to patients. In other embodiments, no fluid is delivered and the nozzle is used to deliver ultrasonic energy alone (in the absence of a spray or other coupling agent) from a non-contact distance.

FIGS. 7-10 provide a brief description of non-contact ultrasound therapy and exemplary nozzle designs. The locking device described herein can be interfitted to any applicator nozzle to prevent re-use of the applicator nozzle. Accordingly, the locking device prevents applicator re-use, thereby preventing possible contamination of wounds and increasing the safety and efficacy of treatment.

Moreover, applicator nozzles are merely exemplary of removable device components with which the locking device of the present invention can be used.

Those skilled in the art will know or be able to ascertain using no more than routine experimentation, many equivalents to the embodiments and practices described herein. Accordingly, it will be understood that the foregoing descriptions are to be considered in all respects illustrative, rather than limiting, of the invention. For example, a variety of systems and/or methods may be implemented based on the disclosure and still fall within the scope of the invention. The specifications and other disclosures in the patents, patent applications, and other references cited herein are hereby incorporated by reference in their entirety. The invention contemplates suitable combinations of one or more of any of the foregoing features of the locking device and/or applicator nozzle.

What is claimed:

1. A locking device, comprising:
a housing having a first sidewall and a second sidewall;
a first arm attached to the first sidewall and having a paddle portion;
a second arm attached to the second sidewall and having a hook portion;
wherein the hook portion is configured to hold the paddle portion in a first position, such that when the hook portion is released, the paddle portion moves to a second position.

2. The apparatus of claim 1, further comprising a third arm, wherein, when the paddle portion is in the second position, the third arm extends beneath the paddle portion and prevents movement of the paddle portion back to the first position.

3. The apparatus of claim 2, wherein the third arm is tapered.

4. The apparatus of claim 3, wherein the paddle portion further comprises a tapered protrusion, wherein, when the paddle portion is in the second position, the tapered third arm extends beneath the paddle portion and creates an interlock connection with the tapered protrusion of the paddle portion.

5. The apparatus of claim 2, wherein the locking device is an apparatus for preventing re-use of a removable component of a medical device, and wherein when the hook portion is released, the paddle portion moves to a second position and prevents re-use of the removable component of the medical device.

6. An apparatus for preventing re-use of an applicator comprising:
a housing having a first sidewall and a second sidewall;
a first arm attached to the first sidewall and having a paddle portion;
a second arm attached to the second sidewall and having a hook portion;
wherein the hook portion is configured to hold the paddle portion in a first position, such that when the hook portion is released, the paddle portion moves to a second position and prevents re-use of the applicator.

7. The apparatus of claim 6, further comprising a third arm, wherein, when the paddle portion is in the second position, the third arm extends beneath the paddle portion and prevents movement of the paddle portion back to the first position.

8. The apparatus of claim 7, wherein the third arm is tapered.

9. The apparatus of claim 8, wherein the paddle portion further comprises a tapered protrusion, wherein, when the paddle portion is in the second position, the tapered third arm extends beneath the paddle portion and creates an interlock connection with the tapered protrusion of the paddle portion.

10. The apparatus of claim 6, further comprising a catch, wherein the hook portion couples with the catch in the first position.

11. The apparatus of claim 6, further comprising a catch and a second catch, wherein the hook portion is positioned between the catch and the second catch in the first position.

12. The apparatus of claim 11, wherein the hook portion moves over the second catch and is positioned against the second catch in the second position.

13. The apparatus of claim 6, further comprising a spring positioned under the first arm to move the first arm to the second position.

14. The apparatus of claim 13, wherein the spring is a leaf spring.

15. The apparatus of claim 6, wherein the apparatus is configured to couple with an ultrasound therapy device.

16. The apparatus of claim 6, wherein the apparatus includes an aperture, and, when the paddle portion is in a second position, the paddle portion extends through the aperture.

17. The apparatus of claim 6, further comprising a base support positioned adjacent to the paddle portion, wherein the base support couples with the paddle portion when the paddle portion is in the second position and prevents the paddle portion from moving past the second position.

18. The apparatus of claim 6, wherein the apparatus is interfitted to an applicator.

19. The apparatus of claim 6, wherein the first arm is attached to the first sidewall at a first point, and the first arm is pivotable about the first point.

20. The apparatus of claim 6, wherein the second arm is attached to the second sidewall at a second point, and the first arm is pivotable about the second point.

21. The apparatus of claim 6, wherein the first arm is a cantilever spring.

22. The apparatus of claim 6, wherein the second arm is a cantilever spring.

23. An applicator for use in treating a wound and modified to prevent re-use, comprising:
a nozzle including a proximal portion and a distal opening, the proximal portion being engagable with a portion of an ultrasound wound therapy device;
wherein said nozzle is interconnected to the apparatus of claim 6.

24. The application of claim 23, wherein the distal opening of the nozzle is configured to allow at least a portion of a transducer tip of the ultrasound wound therapy device to pass therethrough.

25. An applicator for use in treating a wound and modified to prevent re-use, the applicator comprising:
a nozzle including
a proximal portion, a distal opening and a valve interface, the proximal portion being engagable with a portion of an ultrasound wound therapy device, the distal opening allowing at least a portion of a transducer tip of the ultrasound wound therapy device to pass therethrough, the valve interface defining an axis therethrough;
a valve comprising a valve opening, an upper portion and a lower portion, the lower portion being engagable with the valve interface of the nozzle, and the valve opening selectively allows fluid to flow therethrough; and
an aperture, the aperture in fluid communication with at least the upper portion of the valve; and
a device for preventing re-use of said nozzle comprising:
a housing having a first sidewall and a second sidewall;
a first arm attached to the first sidewall and having a paddle portion;
a second arm attached to the second sidewall and having a hook portion;
wherein the hook portion is configured to hold the paddle portion in a first position, such that when the hook portion is released, the paddle portion moves to a second position and prevents re-use of the applicator.

26. The applicator of claim 25, wherein the nozzle is interconnected to the device for preventing re-use.

27. The applicator of claim 25, wherein the applicator further includes a cup including an aperture, the aperture in fluid communication with at least the upper portion of the valve.

28. The applicator of claim 27, wherein fluid flows through the aperture of the cup, through the valve and onto at least a portion of the transducer tip of the ultrasound wound therapy device.

29. A method of preventing re-use of an applicator comprising the steps of:
providing an applicator, comprising:
a nozzle including a proximal portion, a distal opening and a valve interface, the proximal portion being engagable with a portion of an ultrasound wound therapy device, the distal opening allowing at least a portion of a transducer tip of the ultrasound wound therapy device to pass therethrough, the valve interface defining an axis therethrough;
a valve comprising a valve opening, an upper portion and a lower portion, the lower portion being engagable with the valve interface of the nozzle, and the valve opening selectively allows fluid to flow therethrough; and
an aperture, the aperture in fluid communication with at least the upper portion of the valve; and
providing a device for preventing re-use of said nozzle comprising:
a housing having a first sidewall and a second sidewall;
a first arm attached to the first sidewall and having a paddle portion;
a second arm attached to the second sidewall and having a hook portion;
wherein the hook portion is configured to hold the paddle portion in a first position, such that when the hook portion is released, the paddle portion moves to a second position and prevents re-use of the applicator.

30. A kit comprising,
an applicator for use in treating a wound using an ultrasound wound care device and
an apparatus to prevent re-use of the applicator according to claim 6.

31. The kit of claim 30, wherein said applicator and said apparatus to prevent re-use are interconnected.
